(19) United States
(12) Patent Application Publication
(10) Pub. No.: US 2026/0325851 A1
(43) Pub. Date: Oct. 22, 2026

(54) DISCRIMINATING BETWEEN LEFT BUNDLE BRANCH AREA PACING AND VENTRICULAR SEPTAL PACING

(71) Applicant: Medtronic, Inc., Minneapolis, MN (US)

(72) Inventors: Jian Cao, San Jose, CA (US); Xiaohong Zhou, Woodbury, MN (US); Hongyang Lu, Shanghai (CN); Tianyi Shi, Shanghai (CN); Keara A. Berlin, Minneapolis, MN (US); Wade M. Demmer, Coon Rapids, MN (US)

(21) Appl. No.: 18/997,116

(22) PCT Filed: Jul. 28, 2022
(86) PCT No.: PCT/CN2022/108497

§ 371 Date: Jan. 20, 2025

(51) Int. Cl. A61N 1/368; A61N 1/37

(52) U.S. Cl. CPC A61N 1/36842; A61N 1/3712

(57) ABSTRACT

Processing circuitry (50) of a medical system is configured to determine a ventricular activation time metric and determine, based on the ventricular activation time metric, whether ventricular capture includes left bundle branch area pacing or septal pacing. The processing circuitry (50) is further configured to modify a magnitude of the ventricular pacing in response to the ventricular capture including septal pacing.

### FIELD

The disclosure relates generally to medical device systems.

### BACKGROUND

Medical devices may be used to monitor physiological signals of a patient. For example, some medical devices are configured to sense cardiac electrogram (EGM) signals indicative of the electrical activity of the heart via electrodes. Some medical devices may be configured to deliver a therapy in conjunction with or separate from the monitoring of physiological signals. For example, cardiac pacing is an effective therapy for treating patients with bradycardia due to sinus node dysfunction or atrioventricular block. However, traditional right ventricular apical pacing (RVAP) can cause electrical and mechanical dyssynchrony.

### SUMMARY

Conduction system pacing, such as left bundle branch area pacing (LBBAP) therapy, is a physiological pacing approach that activates the normal cardiac conduction and provides synchronized contraction of ventricles. LBBAP therapy may include, but is not limited to, left bundle branch (LBB) pacing and left ventricle (LV) septal pacing that is sufficient to capture LBB fibers. Some medical device systems may lack features enabling the monitoring and modifying of LBBAP therapy delivery, which may be necessary to ensure that LBBAP therapy is appropriately maintained for a patient.

In accordance with techniques of this disclosure, processing circuitry of a medical device system may be configured to detect ventricular capture transition between physiological LBBAP and less desirable septal pacing at different pacing magnitudes, e.g., voltages, and generate output based on the transition pacing threshold. For example, the processing circuitry may determine a ventricular activation time metric and/or a morphological metric. The processing circuitry may determine, based on the morphological metric and/or the ventricular activation time metric, whether ventricular capture includes LBBAP or septal pacing. In some examples, the processing circuitry may then output report to a user including an indication of whether ventricular capture transitioned between LBBAP and septal pacing and/or a pacing magnitude associated with the transition. In some examples, the processing circuitry may automatically modify a magnitude of the ventricular pacing based on the transition between LBBAP and septal pacing. As contrasted with LBBAP, septal pacing includes pacing of a ventricular septum that is not sufficient to capture LBB fibers.

In an example, a medical device system comprises: a medical device configured to deliver ventricular pacing to a heart of a patient; and processing circuitry configured to: receive cardiac electrogram data of a patient sensed by the medical device during delivery of ventricular pacing; identify a plurality of paced heartbeats within the cardiac electrogram data; for each of the plurality of heartbeats: determine a ventricular activation time metric; and determine, based on the ventricular activation time metric, whether ventricular capture comprises left bundle branch area pacing or septal pacing; and responsive to determining ventricular capture comprises septal pacing, modify a magnitude of the ventricular pacing.

In an example, a method comprises: receiving, by processing circuitry of a medical device system comprising a medical device, cardiac electrogram data of a patient sensed by the medical device during delivery of ventricular pacing; identifying, by the processing circuitry, a plurality of heartbeats within the cardiac electrogram data; for each of the plurality of heartbeats: determining, by the processing circuitry, a ventricular activation time metric; and determining, by the processing circuitry and based on the ventricular activation time metric, whether ventricular capture comprises left bundle branch area pacing or septal pacing; and responsive to determining ventricular capture comprises septal pacing, modifying, by the processing circuitry, a magnitude of the ventricular pacing.

In an example, an implantable medical device comprises: therapy delivery circuitry configured to deliver ventricular pacing to a heart of a patient; sensing circuitry configured to sense a cardiac electrogram data of the patient during delivery of ventricular pacing; processing circuitry configured to: identify a plurality of heartbeats within the cardiac electrogram data; for each of the plurality of heartbeats: determine a ventricular activation time metric; and determine, based on the ventricular activation time metric, whether ventricular capture comprises left bundle branch area pacing or septal pacing; and responsive to determining ventricular capture comprises septal pacing, modify a magnitude of the ventricular pacing.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF THE DRAWINGS

FIGS. 1A, 1B and 1C are conceptual diagrams illustrating various example medical systems.

FIG. 2 is a functional block diagram illustrating an example configuration of an implantable medical device (IMD).

FIG. 3 is a functional block diagram illustrating an example configuration of an external device.

FIG. 4 is a block diagram illustrating an example system that includes an access point, a network, external computing devices, such as a server, and one or more other computing devices, which may be coupled to an IMD and external device.

FIG. 5 is a graph illustrating a cardiac EGM including ventricular pacing events and an example technique for determining whether ventricular capture includes left bundle branch area pacing or septal pacing.

FIG. 6 is a conceptual diagram of a depolarization waveform.

FIG. 7 is a flowchart of a method for determining whether ventricular capture includes left bundle branch area pacing or septal pacing in accordance with techniques of this disclosure.

FIG. 8 is a flowchart of a method for determining whether ventricular capture includes left bundle branch area pacing or septal pacing in accordance with techniques of this disclosure.

Like reference characters denote like elements throughout the description and figures.

### DETAILED DESCRIPTION

This disclosure is directed to devices and techniques for classifying pacing captures to evaluate effectiveness of conduction system pacing, e.g., LBBAP. LBBAP therapy may include, but is not limited to, left bundle branch (LBB) pacing and left ventricle (LV) septal pacing that is not sufficient to capture LBB fibers. LBBAP may be with or without direct bundle capture. An example of LBBAP with direct bundle capture may be pacing of the septal tissue near the LBB fibers with sufficient energy to capture the LBB fibers. For instance, if an electrode is in contact with or otherwise near the LBB, less energy may be required to stimulate the LBB, making direct capture of the LBB possible. However, if the electrode is too far away from the LBB fibers, then there may be insufficient energy to capture the LBB, such that only septal pacing is possible.

The classification may be based on cardiac EGMs sensed by the medical device that delivers the conduction system pacing and/or another medical device. A medical device system may include any one or more medical devices configured to sense a cardiac EGM via implanted or external electrodes to implement the techniques of this disclosure for detecting ventricular capture transition between physiological LBBAP and less desirable septal pacing (e.g., LV septal pacing) and vice versa at different pacing voltages. The techniques include evaluation of the cardiac EGM using criteria relating to EGM morphology and ventricular activation time. The techniques of this disclosure for detecting ventricular capture transition between LBBAP and septal pacing may facilitate appropriate maintenance of LBBAP therapy for a patient, in turn leading to improved cardiac wellness.

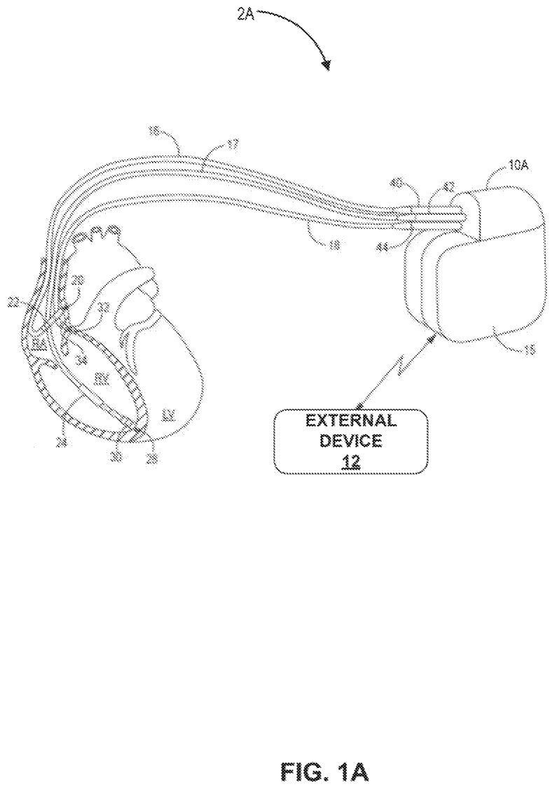

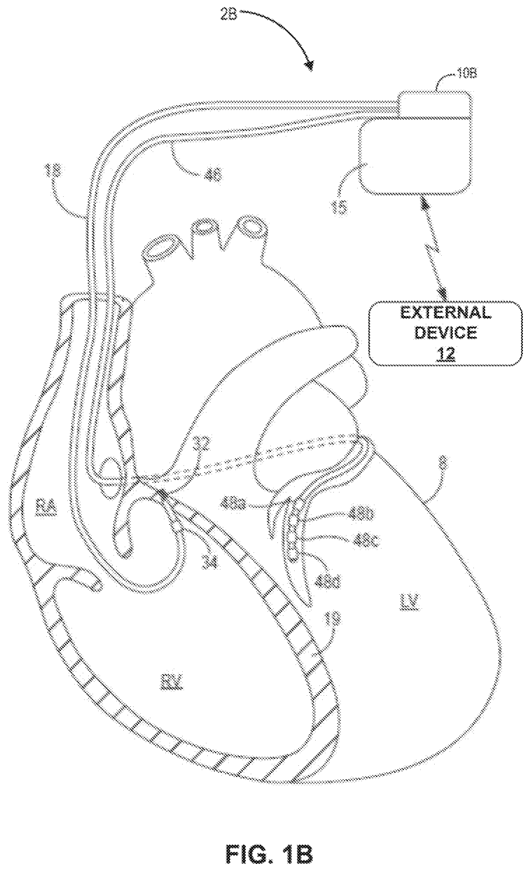

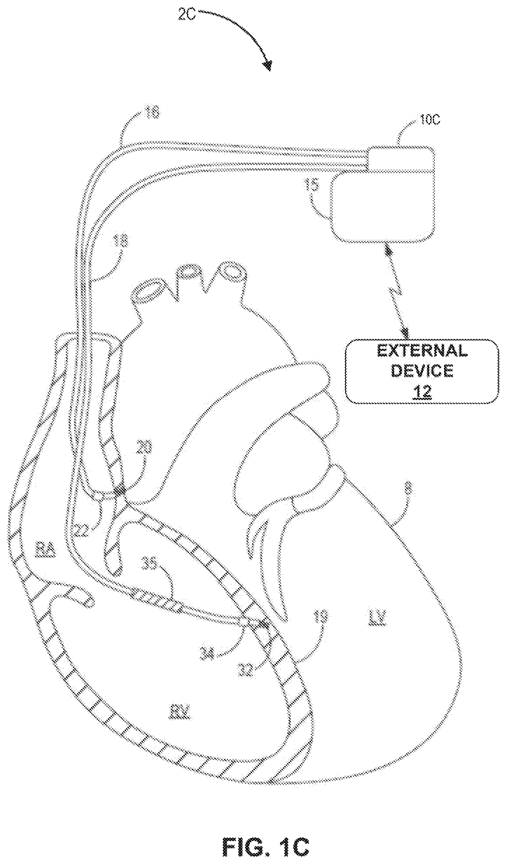

FIGS. 1A-1C illustrate example medical device systems, such as medical device systems 2A-2C (collectively, “medical device system 2”), in accordance with one or more techniques of this disclosure. The example techniques may be used with an implantable medical device (IMD), such as IMDs 10A-10C (collectively, “IMD 10”) shown in FIGS. 1A-1C. Medical device system 2 may include IMD 10 and an external device 12. IMD 10 may be in wireless communication with at least one of external device 12 and other devices not pictured in FIG. 1. IMD 10 may be implanted within a patient, and coupled to one or more implantable medical leads. IMD 10 may be configured to sense one or more cardiac EGMs and deliver conduction system pacing, such as LBBAP, via the plurality of electrodes.

External device 12 may be a computing device with a display viewable by the user and an interface for providing input to external device 12 (i.e., a user input mechanism). In some examples, external device 12 may be a notebook computer, tablet computer, workstation, one or more servers, cellular phone, personal digital assistant, or another computing device that may run an application that enables the computing device to interact with IMD 10. In some examples, external device 12 may be implemented as a number of computing systems configured to receive cardiac electrical signals, which may include at least one EGM signal input received directly or indirectly from IMD 10. In some examples, external device 12 is a computing device included in a remote patient monitoring system such as a computing device included in the CARELINK™ monitoring system available from Medtronic, Inc., Minneapolis, Minnesota.

External device 12 is configured to communicate with IMD 10 and, optionally, another computing device (not illustrated in FIG. 1), via wireless communication. External device 12, for example, may communicate via near-field communication technologies (e.g., inductive coupling, NFC or other communication technologies operable at ranges less than 10-20 cm) and far-field communication technologies (e.g., RF telemetry according to the 802.11 or Bluetooth® specification sets, or other communication technologies operable at ranges greater than near-field communication technologies).

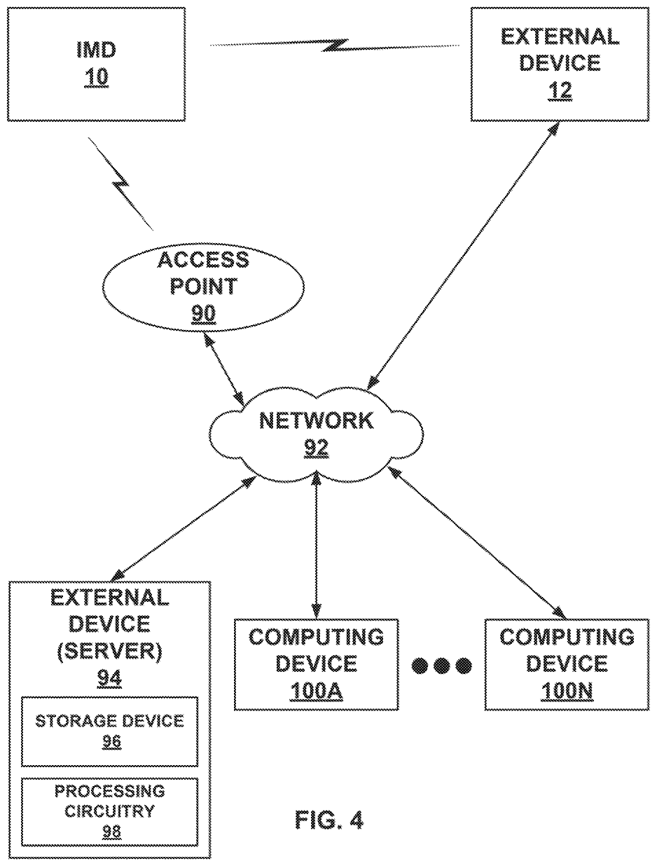

External device 12 may be used to configure operational parameters for IMD 10. External device 12 may be used to retrieve data from IMD 10. The retrieved data may include values of physiological parameters measured by IMD 10, indications of episodes of arrhythmia or other maladies detected by IMD 10, and physiological signals recorded by IMD 10. For example, external device 12 may retrieve information related to detection of ventricular capture transitioning between LBBAP and septal pacing, e.g., LV septal pacing, by IMD 10, such as a count or other quantification of such transitions (e.g., over a time period since the last retrieval of information by external device), and the pacing magnitudes associated with such transitions. External device 12 may also retrieve cardiac EGM segments (e.g., windows of data) recorded by IMD 10 (e.g., due to IMD 10 determining that an episode of arrhythmia or another malady occurred during the segment, or in response to a request to record the segment from the patient or another user). As discussed in greater detail below with respect to FIG. 4, one or more remote computing devices may interact with IMD in 10 in a manner similar to external device 12, e.g., to program IMD 10 and/or retrieve data from IMD 10, via a network.

Processing circuitry of medical device system 2 (e.g., of IMD 10, external device 12, and/or of one or more other computing devices) may be configured to perform the example techniques of this disclosure for detecting ventricular capture transitioning between LBBAP and septal pacing. In some examples, the processing circuitry of system 2 analyzes a cardiac EGM sensed by IMD 10 during delivery of ventricular pacing to determine whether ventricular capture includes LBBAP or septal pacing based on a morphological metric and a ventricular activation time metric. The morphological metric and/or the ventricular activation time metric are described in greater detail below. In brief, determining the morphological metric may include comparing a segment of the cardiac electrogram data associated with a heartbeat to a template and determining the morphological metric based on the comparison. Determining the ventricular activation time metric may include determining a center of area under a segment of a depolarization waveform and a maximum ventricular amplitude time for a heartbeat and determining the ventricular activation time metric for the heartbeat based on the center of area and the maximum ventricular amplitude time.

FIG. 1A is a conceptual diagram of medical device system 2A capable of pacing and sensing in a patient's heart 8. System 2A may include IMD 10A coupled to a heart 8 via transvenous electrical leads 16, 17 and 18. IMD 10A is shown in FIG. 1A as a dual chamber device configured to deliver cardiac pacing pulses and sensing cardiac electrical signals from the right atrium (RA) and from the ventricular chambers. Housing 15 may enclose internal circuitry corresponding to various circuits and components for sensing cardiac signals from heart 8 and delivering cardiac pacing therapy. In some examples, IMD 10A may be configured to detect tachyarrhythmias from the sensed cardiac signals and deliver high voltage cardioversion/defibrillation (CV/DF) shocks to heart 8, e.g., for terminating a detected ventricular tachycardia or ventricular fibrillation.

Lead 16 may be a RA lead (“RA lead 16”), and lead 18 may be a ventricular conduction system pacing lead (“VCS pacing lead 18). RA lead 16 and VCS pacing lead 18 may be advanced transvenously to position electrodes for sensing cardiac electrical signals and delivering pacing therapy. RA lead 16 may be positioned such that its distal end is in the vicinity of the right atrium. RA lead 16 may carry pacing and sensing electrodes 20 and 22, shown as a tip electrode and a ring electrode, respectively, spaced proximally from tip electrode 20. Electrodes 20 and 22 provide sensing and pacing in the RA and are each connected to a respective insulated conductor extending within the elongated body of RA lead 16. Each insulated conductor is coupled at its proximal end to a connector carried by proximal lead connector 40. IMD 10A may include a connector block configured to receive lead connector 40 for electrically coupling conductors extending from the distal electrodes 20 and 22 to circuitry within housing 15 via electrical feedthroughs crossing housing 15.

VCS lead 18 may be advanced within the right atrium to position a tip electrode 32 and a ring electrode 34 for pacing and sensing in the vicinity of the VCS, e.g., at or near the His bundle, from a right atrial approach, as shown. VCS tip electrode 32 may be a helical electrode that is advanced into the inferior end of the interatrial septum, beneath the AV node and near the tricuspid valve annulus to position tip electrode 32 in, along or proximate to the His bundle. Ring electrode 34 spaced proximally from tip electrode 32 may be used as the return electrode with the cathode tip electrode 32 for pacing the right and left ventricles via the native His-Purkinje conduction system.

IMD 10A may produce an intracardiac electrogram (EGM) signal from the cardiac electrical signal received via a sensing electrode vector that may include tip electrode 32 and/or ring electrode 34 of VCS lead 18. The electrodes 32 and 34 are coupled to respective insulated conductors extending within the elongated body of VCS lead 18, which provide electrical connection to the proximal lead connector 44 coupled to circuitry of IMD 10A.

Housing 15 may function as a return electrode for unipolar sensing or pacing configurations with any of the electrodes carried by leads 16 and 18. Electrodes 32 and 34 may be used in a bipolar pacing electrode pair for delivering VCS pacing pulses and for receiving a cardiac electrical signal for sensing intrinsic and pacing evoked QRS waveforms. In some examples, IMD 10A may be configured to sense a far field (FF) cardiac signal, e.g., using electrode 32 and housing 15 or using electrode 34 and housing 15, and/or a near field (NF) cardiac signal, e.g., using electrodes 32 and 34, for processing and analysis for determining a capture type. Electrodes 32 and 34 may be used in a sensing electrode vector for sensing intrinsic R-waves for use in determining a heart rhythm and a need for electrical stimulation therapy.

In addition to pacing and sensing capabilities, IMD 10A may be capable of delivering high voltage cardioversion or defibrillation (CV/DF) shocks in some examples. In this case, IMD 10A may be coupled to at least one lead carrying one or more defibrillation electrodes, which may be elongated coil electrodes used to deliver high voltage CV/DF shocks. In the example of FIG. 1A, a third lead 17 is shown coupled to IMD 10A with a distal end advanced into the right ventricle (RV). RV lead 17 may include a coil electrode 24 used for delivering CV/DF shocks, e.g., in combination with housing 15. Housing 15 may function as an active electrode during CV/DF shock delivery in conjunction with coil electrode 24. RV lead 17, when present, may carry a distal tip electrode 28 and ring electrode 30 for delivering ventricular pacing pulses in the RV and/or sensing a cardiac electrical signal from the RV. The electrodes 28, 30 and 24 are each connected to a respective insulated conductor extending within the elongated body of RV lead 17. Each insulated conductor may be coupled at its proximal end to a connector carried by proximal lead connector 42. In other examples, RV lead 17 is optional. When IMD 10A is implemented as an implantable cardioverter defibrillator (ICD) with CV/DF shock capabilities in addition to cardiac pacing and sensing functions, one or more CV/DF electrodes may be carried by RA lead 16 and/or VCS lead 18.

It is to be understood that although IMD 10A is described as a dual chamber pacemaker capable of sensing and pacing the RA and sensing ventricular signals and pacing the ventricular chambers via the VCS, with or without CV/DF shock capabilities, IMD 10A may be a single chamber pacing device with single chamber or dual chamber sensing. For example, IMD 10A may be coupled only to VCS lead 18 for sensing cardiac electrical signals and delivering VCS pacing pulses for at least maintaining a minimum ventricular rate. VCS lead 18 may carry additional sensing electrodes positioned within the RA for sensing a RA EGM signal when lead 18 is positioned for delivering VCS pacing pulses such that IMD 10A is capable of dual chamber (atrial P-wave and ventricular R-wave) sensing and VCS pacing, which may be atrial synchronous ventricular pacing delivered via the His-Purkinje system.

FIG. 1B is a conceptual diagram of medical device system 2B. System 2B may include IMD 10B coupled to VCS lead 18 advanced to an alternative location within the heart 8 for delivering VCS pacing pulses and sensing cardiac electrical signals. In this example, the distal portion of VCS lead 18 is advanced within the right ventricle (RV) for sensing cardiac electrical signals and delivering VCS pacing pulses to or in the vicinity of the His bundle from a right ventricular approach. IMD 10B may be a single chamber device coupled only to VCS lead 18. In other examples, IMD 10B may be a dual chamber device and be coupled to RA lead 16 (shown in FIG. 1A) and VCS lead 18, to enable sensing of atrial P-waves and delivery of atrial pacing pulses and delivery of VCS pacing pulses at an AV delay from atrial events, sensed or paced, in an atrioventricular synchronous pacing mode.

In the example of FIG. 1B, the tip electrode 32 of VCS lead 18 is placed in the inter-ventricular septum 19, e.g., high along the inter-ventricular septum near the inferior portion of the His bundle. Tip electrode 32 may be paired with the return anode ring electrode 34 for delivering VCS pacing pulses and for sensing raw cardiac electrical signals, which may be processed for obtaining an NF EGM signal. A post-pace NF EGM signal may be provided as input to PCC 51 in some examples for classifying capture type using the techniques disclosed herein. The tip electrode 32 or the ring electrode 34 may be paired with IMD housing 15 for receiving a raw cardiac electrical signal that is processed to obtain an FF EGM signal.

In some examples, VCS pacing may be delivered in combination with LV myocardial pacing that can be delivered via an LV lead 46 for further improvement in mechanical synchrony of the RV and LV, e.g., during cardiac resynchronization therapy (CRT). LV lead 46 may be advanced into the RA, through the coronary sinus ostium and into a cardiac vein of the LV for positioning electrodes 48a, 48b, 48c and 48d (collectively “LV electrodes 48”) along the LV myocardium for sensing ventricular electrical signals and pacing the LV myocardium. LV lead 46 is shown in FIG. 1B as a quadripolar lead carrying four electrodes 48a-d that may be selected in various bipolar pacing electrode pairs for pacing the LV myocardial tissue and for sensing LV signals. One of LV electrodes 48 may be selected in combination with pacemaker housing 15 for delivering unipolar LV myocardial pacing in some instances and/or for sensing a raw cardiac electrical signal that may be processed and analyzed as an FF EGM signal.

When VCS lead 18 is positioned for delivering His bundle or bundle branch pacing of one or both bundle branches, VCS pacing may be combined with ventricular myocardial pacing of the LV (using LV lead 46) to correct an LV conduction delay and achieve electrical and mechanical synchrony of the ventricles. As such, in some examples, IMD 10B may control VCS pacing pulse delivery in combination with LV myocardial pacing pulse delivery at specified time intervals which may include an AV delay and/or an VV delay. The AV delay may control the timing of the VCS pulses and/or the LV myocardial pacing pulses relative to an atrial event, e.g., sensed P-wave or delivered atrial pacing pulse. In some examples, a VV delay may control the timing between a VCS pacing pulse delivered via VCS lead 18 and an LV myocardial pacing pulse delivered via LV lead 46.

It is to be understood that LV lead 46 is optional. In some examples, IMD 10B is coupled only to VCS lead 18 advanced into the RA or the RV for delivering VCS pacing and sensing ventricular EGM signals. In other examples, RA lead 16 shown in FIG. 1A is implanted in combination with the VCS lead 18 advanced into the RA or the RV for delivering VCS pacing in a dual chamber sensing and pacing system. External device 50 may receive one or more EGM signals from IMD 10B sensed using any available ventricular EGM sensing electrode vector for processing and analysis according to the techniques disclosed herein.

FIG. 1C is a conceptual diagram of medical device system 2C. System 2C may include IMD 10C. In the example of FIG. 1C, IMD 10C includes VCS lead 18 coupled to IMD 10C. In FIG. 1C, IMD 10C is a dual chamber device configured to receive RA lead 16, positioned in the right atrial chamber for delivering atrial pacing pulses and sensing atrial electrical signals via electrodes 20 and 22. IMD 10C may be configured to sense intrinsic atrial P-waves and deliver atrial pacing pulses in the absence of sensed P-waves. IMD 10C may be configured to deliver atrial synchronized ventricular pacing by setting an AV delay in response to each sensed P-wave or delivered atrial pacing pulse and deliver a ventricular pacing pulse to the VCS via lead 18 upon the expiration of the AV delay.

VCS lead 18 may be advanced transvenously into the RV via the RA for positioning tip electrode 32 within inter-ventricular septum 19, relatively lower than (inferior to) the implant position shown in FIG. 1B. When tip electrode 32 is advanced relatively superiorly within the inter-ventricular septum 19, as shown in FIG. 1B, tip electrode 32 may be positioned along the inferior portion of the His bundle for delivering pacing pulses for complete or partial capture of the His bundle. In FIG. 1C, tip electrode 32 may be advanced within the inter-ventricular septum 19 in the vicinity of a bundle branch of the His-Purkinje system, e.g., at a LBB pacing site in the area of the LBB or at a right bundle branch RBB pacing site in the area of the RBB, for delivering pacing pulses for capturing one or both bundle branches.

Tip electrode 32 may be selected as a pacing cathode electrode in combination with ring electrode 34 as the return anode electrode for pacing and capturing the LBB and/or RBB in various examples. In some instances, the pacing pulse amplitude and pulse width may be selected to achieve cathodal capture at the cathode electrode for capturing at least one bundle branch. In other instances, the pacing pulse amplitude and pulse width may be selected to achieve cathodal and anodal capture, which may capture both the LBB and the RBB concurrently to provide dual or bilateral bundle branch (BB) pacing using a single bipolar electrode pair. In other examples, either tip electrode 32 or ring electrode 34 may be selected as cathode electrode paired with housing 15 in a unipolar pacing electrode vector. Unipolar pacing may capture a single BB. In some cases, however, unipolar pacing may capture both the RBB and the LBB when a unipolar pacing pulse directly captures one bundle branch while virtual current or break excitation generated by the pacing electrode may excite the other bundle branch, potentially resulting in unipolar bilateral BB pacing, with capture of both the LBB and RBB.

While VCS lead 18 is shown carrying one pacing and sensing electrode pair, tip electrode 32 and ring electrode 34, it is to be understood that in other examples, VCS lead 18 may include multiple electrodes along its distal portion to provide one or more selectable bipolar pacing electrode vectors and/or one or more unipolar pacing electrode vectors (e.g., with housing 15) for delivering pacing pulses to one or both of the RBB and the LBB.

Furthermore, VCS lead 18 may include one or more coil electrodes, e.g., coil electrode 35, when IMD 10C is configured as an ICD capable of delivering high voltage shock therapies. Coil electrode 35 may be used in sensing electrode vectors, e.g., with either of tip electrode 32 or ring electrode 34, for sensing a ventricular EGM signal that may be analyzed by PCC 51 for classifying VCS pacing pulse capture type.

As described above, IMD 10C may communicate via wireless telemetry with external device 12. External device 12 may receive EGM signals from IMD 10C sensed using any available electrodes shown in FIG. 1C or in other examples described or shown in the accompanying drawings for processing and analysis according to the techniques disclosed herein.

Although IMD 10 is generally described herein as a pacemaker that delivers conduction system pacing and determines whether the pacing results in effective conduction system pacing, example systems including one or more implantable or external devices of any type configured to sense a cardiac EGM may be configured to implement the techniques of this disclosure.

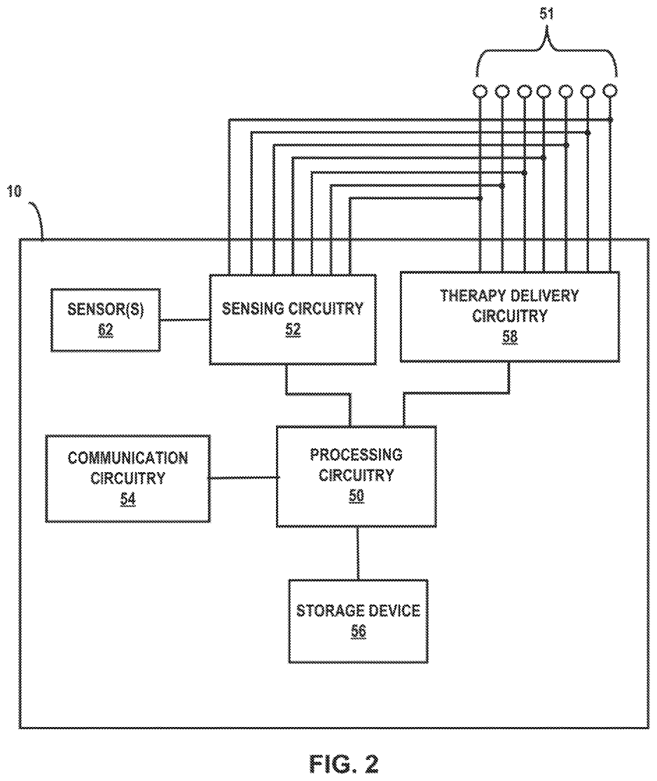

FIG. 2 is a functional block diagram illustrating an example configuration of IMD 10 of FIG. 1 in accordance with one or more techniques described herein. In the illustrated example, IMD 10 includes electrodes 51, which may be located on one or more leads (e.g., lead 16) and/or a housing (e.g., housing 15) of IMD 10, processing circuitry 50, sensing circuitry 52, communication circuitry 54, storage device 56, therapy delivery circuitry 58, and sensors 62. Electrodes 51 may include electrodes described herein, such as electrodes 20, 22, 28, 30, 32, 34, 48, etc.

Processing circuitry 50 may include fixed function circuitry and/or programmable processing circuitry. Processing circuitry 50 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more DSPs, one or more ASICs, or one or more FPGAs, as well as other discrete or integrated logic circuitry. The functions attributed to processing circuitry 50 herein may be embodied as software, firmware, hardware or any combination thereof.

Sensing circuitry 52 may be coupled to electrodes 51. Sensing circuitry 52 may sense signals from electrodes 51, e.g., to produce a cardiac EGM, in order to facilitate monitoring the electrical activity of the heart. Sensing circuitry 52 also may monitor signals from sensors 62, which may include one or more accelerometers, pressure sensors, and/or optical sensors, as examples. In some examples, sensing circuitry 52 may include one or more filters and amplifiers for filtering and amplifying signals received from electrodes 51 and/or sensors 62. Sensing circuitry 52 may include switching circuitry for selecting electrodes 51 and their polarity for sensing the cardiac EGM signals described herein.

Sensing circuitry 52 and/or processing circuitry 50 may be configured to detect cardiac depolarizations (e.g., P-waves of atrial depolarizations or R-waves of ventricular depolarizations) when the cardiac EGM amplitude crosses a sensing threshold. For cardiac depolarization detection, sensing circuitry 52 may include a rectifier, filter, amplifier, comparator, and/or analog-to-digital converter, in some examples. In some examples, sensing circuitry 52 may output an indication to processing circuitry 50 in response to sensing of a cardiac depolarization. In this manner, processing circuitry 50 may receive detected cardiac depolarization indicators corresponding to the occurrence of detected R-waves and P-waves in the respective chambers of heart. Processing circuitry 50 may use the indications of detected R-waves and P-waves for determining inter-depolarization intervals, heart rate, and detecting arrhythmias, such as tachyarrhythmias and asystole.

Sensing circuitry 52 may also provide one or more digitized cardiac EGM signals to processing circuitry 50 for analysis. For example, processing circuitry 50 may use the digitized cardiac EGM signals in cardiac rhythm discrimination and/or to determine whether ventricular capture transitioned between LBBAP and septal pacing according to the techniques of this disclosure. In some examples, processing circuitry 50 may store the digitized cardiac EGM in storage device 56. Processing circuitry 50 of IMD 10, and/or processing circuitry of another device that retrieves data from IMD 10, may analyze the cardiac EGM to determine whether ventricular capture transitioned between LBBAP and septal pacing according to the techniques of this disclosure.

Communication circuitry 54 may include any suitable hardware, firmware, software or any combination thereof for communicating with another device, such as external device 12, another networked computing device, or another IMD or sensor. Under the control of processing circuitry 50, communication circuitry 54 may receive downlink telemetry from, as well as send uplink telemetry to external device 12 or another device with the aid of an internal or external antenna. In addition, processing circuitry 50 may communicate with a networked computing device via an external device (e.g., external device 12) and a computer network, such as the Medtronic CareLink® Network. Communication circuitry 54 may be configured to transmit and/or receive signals via inductive coupling, electromagnetic coupling, Near Field Communication (NFC), Radio Frequency (RF) communication, Bluetooth, WiFi, or other proprietary or non-proprietary wireless communication schemes.

In some examples, storage device 56 includes computer-readable instructions that, when executed by processing circuitry 50, cause IMD 10 and processing circuitry 50 to perform various functions attributed to IMD 10 and processing circuitry 50 herein. Storage device 56 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media. Storage device 56 may store, as examples, programmed values for one or more operational parameters of IMD 10 and/or data collected by IMD 10 for transmission to another device using communication circuitry 54. Data stored by storage device 56 and transmitted by communication circuitry 54 to one or more other devices may include digitized cardiac EGMs, and data as described herein regarding transitions between LBBAP and septal pacing, as examples.

Processing circuitry 50 may be configured to control therapy delivery circuitry 58 to generate and deliver electrical therapy to heart 8 of the patient via electrodes 51. Electrical therapy may include, for example, pacing pulses, or any other suitable electrical stimulation. Processing circuitry 50 may control therapy delivery circuitry 58 to deliver electrical stimulation therapy via electrodes 51 according to one or more therapy parameter values, which may be stored in storage device 56. The therapy parameter values may, in the case of pacing pulses, include magnitude values, such as pulse amplitude (e.g., a stimulation voltage amplitude) and width. Therapy delivery circuitry 58 may include capacitors, current sources, and/or regulators, in some examples. Therapy delivery circuitry 58 may include switching circuitry for selecting electrodes 51 and their polarity for delivering therapy signals to heart 8.

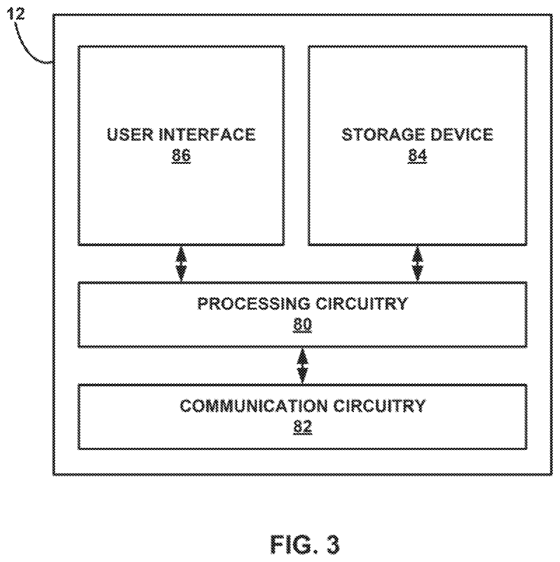

FIG. 3 is a block diagram illustrating an example configuration of components of external device 12. In the example of FIG. 3, external device 12 includes processing circuitry 80, communication circuitry 82, storage device 84, and user interface 86.

Processing circuitry 80 may include one or more processors that are configured to implement functionality and/or process instructions for execution within external device 12. For example, processing circuitry 80 may be capable of processing instructions stored in storage device 84. Processing circuitry 80 may include, for example, microprocessors, DSPs, ASICS, FPGAs, or equivalent discrete or integrated logic circuitry, or a combination of any of the foregoing devices or circuitry. Accordingly, processing circuitry 80 may include any suitable structure, whether in hardware, software, firmware, or any combination thereof, to perform the functions ascribed herein to processing circuitry 80.

Communication circuitry 82 may include any suitable hardware, firmware, software or any combination thereof for communicating with another device, such as IMD 10. Under the control of processing circuitry 80, communication circuitry 82 may receive downlink telemetry from, as well as send uplink telemetry to, IMD 10, or another device. Communication circuitry 82 may be configured to transmit or receive signals via inductive coupling, electromagnetic coupling, Near Field Communication (NFC), Radio Frequency (RF) communication, Bluetooth, WiFi, or other proprietary or non-proprietary wireless communication schemes. Communication circuitry 82 may also be configured to communicate with devices other than IMD 10 via any of a variety of forms of wired and/or wireless communication and/or network protocols.

Storage device 84 may be configured to store information within external device 12 during operation. Storage device 84 may include a computer-readable storage medium or computer-readable storage device. In some examples, storage device 84 includes one or more of a short-term memory or a long-term memory. Storage device 84 may include, for example, RAM, DRAM, SRAM, magnetic discs, optical discs, flash memories, or forms of EPROM or EEPROM. In some examples, storage device 84 is used to store data indicative of instructions for execution by processing circuitry 80. Storage device 84 may be used by software or applications running on external device 12 to temporarily store information during program execution.

Data exchanged between external device 12 and IMD 10 may include operational parameters. External device 12 may transmit data including computer readable instructions which, when implemented by IMD 10, may control IMD 10 to change one or more operational parameters and/or export collected data. For example, processing circuitry 80 may transmit an instruction to IMD 10 which requests IMD 10 to export collected data (e.g., transition detection data and/or digitized cardiac EGMs) to external device 12. In turn, external device 12 may receive the collected data from IMD 10 and store the collected data in storage device 84. Processing circuitry 80 may implement any of the techniques described herein to analyze cardiac EGMs received from IMD 10, e.g., to determine whether ventricular capture transitioned between LBBAP and septal pacing according to the techniques of this disclosure. In some examples, processing circuitry 80 of external device 12 may initiate and the control the performance of the testing of various pacing magnitudes to identify transitions between LPPAP and septal pacing

A user, such as a clinician or the patient, may interact with external device 12 through user interface 86. User interface 86 includes a display (not shown), such as a liquid crystal display (LCD) or a light emitting diode (LED) display or other type of screen, with which processing circuitry 80 may present information related to IMD 10, e.g., cardiac EGMs, indications of detections of ventricular capture transitioning between LBBAP and septal pacing. In addition, user interface 86 may include an input mechanism to receive input from the user. The input mechanisms may include, for example, any one or more of buttons, a keypad (e.g., an alphanumeric keypad), a peripheral pointing device, a touch screen, or another input mechanism that allows the user to navigate through user interfaces presented by processing circuitry 80 of external device 12 and provide input. In other examples, user interface 86 also includes audio circuitry for providing audible notifications, instructions or other sounds to the user, receiving voice commands from the user, or both.

FIG. 4 is a block diagram illustrating an example system that includes an access point 90, a network 92, external computing devices, such as a server 94, and one or more other computing devices 100A-100N (collectively, “computing devices 100”), which may be coupled to IMD 10 and external device 12 via network 92, in accordance with one or more techniques described herein. In this example, IMD 10 may use communication circuitry 54 to communicate with external device 12 via a first wireless connection, and to communicate with an access point 90 via a second wireless connection. In the example of FIG. 4, access point 90, external device 12, server 94, and computing devices 100 are interconnected and may communicate with each other through network 92.

Access point 90 may include a device that connects to network 92 via any of a variety of connections, such as telephone dial-up, digital subscriber line (DSL), or cable modem connections. In other examples, access point 90 may be coupled to network 92 through different forms of connections, including wired or wireless connections. In some examples, access point 90 may be a user device, such as a tablet or smartphone, that may be co-located with the patient. IMD 10 may be configured to transmit data, such as data regarding the identification of transitions between LBBAP and septal pacing and/or cardiac EGMs, to access point 90. Access point 90 may then communicate the retrieved data to server 94 via network 92.

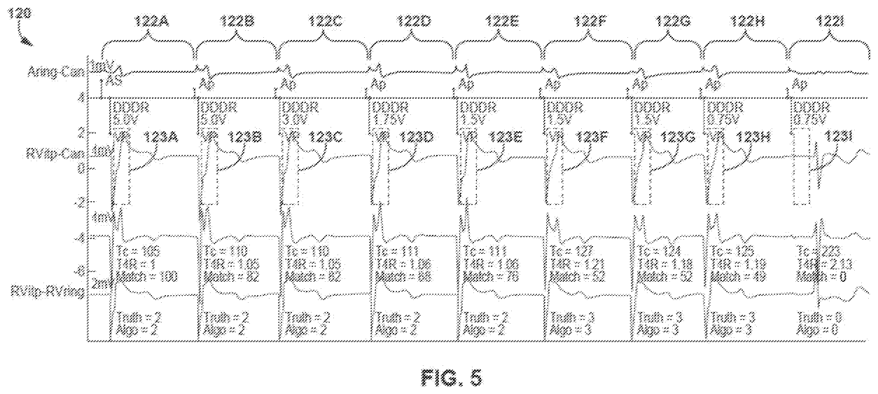

In some cases, server 94 may be configured to provide a secure storage site for data that has been collected from IMD 10 and/or external device 12. In some cases, server 94 may assemble data in web pages or other documents for viewing by trained professionals, such as clinicians, via computing devices 100. One or more aspects of the illustrated system of FIG. 5 may be implemented with general network technology and functionality, which may be similar to that provided by the Medtronic CareLink® Network.

In some examples, one or more of computing devices 100 may be a tablet or other smart device located with a clinician, by which the clinician may program, receive data from, and/or interrogate IMD 10. For example, the clinician may access data collected by IMD 10 through a computing device 100, such as when a patient is in in between clinician visits, to check on a status of a medical condition or the operation of IMD 10. In some examples, the clinician may enter instructions for a medical intervention for the patient into an application executed by computing device 100, such as based on a status of a patient condition determined by IMD 10, external device 12, server 94. or any combination thereof, or based on other patient data known to the clinician. Device 100 then may transmit the instructions for medical intervention to another of computing devices 100 located with the patient or a caregiver of the patient. For example, such instructions for medical intervention may include an instruction to change a drug dosage, timing, or selection, to schedule a visit with the clinician, or to seek medical attention. In further examples, a computing device 100 may generate an alert to the patient based on a status of a medical condition of the patient, which may enable the patient proactively to seek medical attention prior to receiving instructions for a medical intervention. In this manner, the patient may be empowered to take action, as needed, to address his or her medical status, which may help improve clinical outcomes for the patient. In some examples, the clinician may enter instructions for IMD 10 to perform a pacing magnitude test to identify transitions between LBBAP and septal pacing via computing device 100.

In the example illustrated by FIG. 4, server 94 includes a storage device 96, e.g., to store data retrieved from IMD 10, and processing circuitry 98. Although not illustrated in FIG. 4, computing devices 100 may similarly include a storage device and processing circuitry. Processing circuitry 98 may include one or more processors that are configured to implement functionality and/or process instructions for execution within server 94. For example, processing circuitry 98 may be capable of processing instructions stored in memory 96. Processing circuitry 98 may include, for example, microprocessors, DSPs, ASICs, FPGAs, or equivalent discrete or integrated logic circuitry, or a combination of any of the foregoing devices or circuitry. Accordingly, processing circuitry 98 may include any suitable structure, whether in hardware, software, firmware, or any combination thereof, to perform the functions ascribed herein to processing circuitry 98. Processing circuitry 98 of server 94 and/or the processing circuitry of computing devices 100 may implement any of the techniques described herein to analyze cardiac EGMs received from IMD 10, e.g., to determine whether ventricular capture transitioned between LBBAP and septal pacing according to the techniques of this disclosure.

Storage device 96 may include a computer-readable storage medium or computer-readable storage device. In some examples, memory 96 includes one or more of a short-term memory or a long-term memory. Storage device 96 may include, for example, RAM, DRAM, SRAM, magnetic discs, optical discs, flash memories, or forms of EPROM or EEPROM. In some examples, storage device 96 is used to store data indicative of instructions for execution by processing circuitry 98.

FIG. 5 is a graph illustrating an example cardiac EGM 120 over a plurality of paced cardiac cycles 122A-122I (collectively, “cardiac cycles 122”) for which IMD 10 delivered ventricular cardiac pacing at different stimulation magnitudes, e.g., different stimulation voltages. In some examples, cardiac EGM 120 is a far-field ventricular EGM, e.g., between an electrode on lead 16 and an electrode on housing 15 of IMD 10. FIG. 5 also illustrates an example technique for detecting ventricular capture transition between LBBAP and septal pacing based on cardiac EGMs 120. IMD 10 may sense EGMs 120 of the patient during delivery of ventricular pacing (e.g., as part of therapeutic pacing and/or during a threshold detection pacing procedure) to the patient. Processing circuitry 50 of IMD 10 may receive EGM 120 and identify a plurality of heartbeats and features within EGM 120. Processing circuitry 50 may analyze analysis windows 123A-123I (collectively, “analysis windows 123”) for paced cardiac cycles 122 to identify the features within EGM 120.

According to the techniques of this disclosure, processing circuitry, e.g., of IMD 10, uses different features such as ventricular activation time and/or morphology characteristics to detect a change in ventricular capture. IMD 10 senses cardiac EGM 120 and detects features within analysis windows 123 for cardiac cycles 122. For each of analysis windows 123, processing circuitry 50 may begin collecting a window of EGM 120 after delivery of a corresponding pacing voltage to heart 8. In other words, for each of analysis windows 123, processing circuitry may record a digitized far-field EGM for a predetermined period of time shortly after the pace (e.g., to avoid collecting data during the pace). Processing circuitry 50 may, for each of cardiac cycles 122, determine a ventricular activation time metric and/or a morphological metric. Based on the ventricular activation time metric and/or the morphological metric, processing circuitry 50 may determine whether ventricular capture includes LBBAP or septal pacing.

Processing circuitry 50 may determine whether ventricular capture includes LBBAP or septal pacing based on a ventricular activation time metric. In some examples, the ventricular activation time metric may be determined based on electrocardiogram (ECG) data and/or EGM data. In the case of EGM data, parameters in the EGM data may reflect changes in ventricular activation time. For instance, processing circuitry 50 may indirectly measure or generate the ventricular activation time based on an EGM morphological metric. In any case, processing circuitry 50 may determine the ventricular activation time metric for each heartbeat of a plurality of heartbeats, such as each of cardiac cycles 122 of EGM 120. In general, a relatively small ventricular activation time metric value may indicate that ventricular capture includes LBBAP (but not septal pacing), and a relatively large ventricular activation time metric may indicate that ventricular capture includes septal pacing (but not LBBAP).

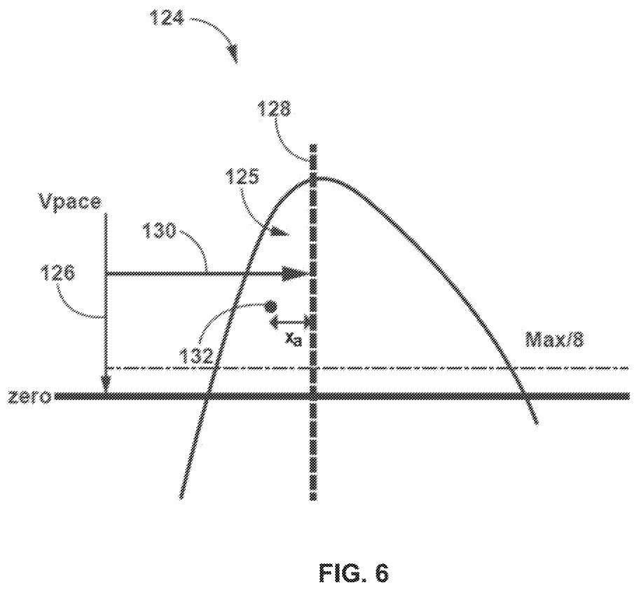

In some examples, processing circuitry 50 may determine the ventricular activation time metric for the heartbeat based on a center of area under a segment of a depolarization waveform and a maximum ventricular amplitude time for the heartbeat (e.g., the time between a ventricular pacing event for a heartbeat and the maximum amplitude for the heartbeat). The center of area under the segment of the ventricular depolarization waveform may represent the centroid or geometric center of the segment of the ventricular depolarization waveform. Accordingly, processing circuitry 50 may determine the center of area using the equation below:

$${X}_{a} = \frac{{\sum }_{i = 1}^{N} i * z ( i )}{{\sum }_{i = 1}^{N} z ( i )}$$

where Xₐ represents the time for the center of area under the segment of the ventricular depolarization waveform, the upper and lower limits of summation correspond to the upper and lower bounds of the domain of the ventricular depolarization waveform, and z(i) represents the amplitude of ventricular depolarization waveform as a function of the index of summation. Additionally or alternatively, the center of area under the segment of the ventricular depolarization waveform may be calculated using any other equation for computing the arithmetic mean position of a geometric figure. Techniques for calculating the center of area are discussed in greater detail below with respect to FIG. 6.

Processing circuitry 50 may determine the ventricular activation time metric for the heartbeat based on a center of area under a segment of a depolarization waveform and a maximum ventricular amplitude time for the heartbeat. In some instances, processing circuitry 50 may determine the ventricular activation time metric using the following equation:

t₄=tₘₐₓ−Xₐ
where t₄ represents one example of the ventricular activation time metric, which may be representative of both the left and right ventricular activation time from far-field EGM, tₘₐₓ represents the maximum ventricular amplitude time, and Xₐ represents the time for the center of area under the segment of the ventricular depolarization waveform. As shown in FIG. 5, in some examples, processing circuitry 50 may calculate a T4R. T4R may represent another example of the ventricular activation time metric. Processing circuitry 50 may calculate T4R by determining a ratio of the respective t₄ for a heartbeat (e.g., cardiac cycle 122C) and the t₄ for a template. The template may represent a window of waveform data collected for one or more heartbeats known to be LBBAP (e.g., due to IMD 10 delivering a high pacing voltage, such as 5V, to heart 8). In other words, processing circuitry 50 may compare cardiac cycles 122 to one or more collected and stored templates of a patient's EGM data associated with heartbeats known or otherwise confirmed to be LBBAP.

A relatively small ventricular activation time metric value may indicate a higher likelihood that ventricular capture includes LBBAP (but not septal pacing), and a relatively large ventricular activation time metric may indicate that ventricular capture includes septal pacing (but not LBBAP). As such, processing circuitry 50 may use an algorithm based on the ventricular activation time metric (e.g., t₄ and/or T4R) to determine whether ventricular capture includes LBBAP or septal pacing. For example, if t₄ and/or T4R values are above respective ventricular activation time thresholds, processing circuitry 50 may determine that ventricular capture includes septal pacing. On the other hand, if t₄ and/or T4R values are below respective ventricular activation time thresholds, processing circuitry 50 may determine that ventricular capture includes LBBAP. An example threshold for t₄ may be a value between about 50 and about 350 milliseconds (ms). An example threshold for T4R threshold may be a value between about 0.8 to 1.5. However, other thresholds are contemplated by this disclosure.

Processing circuitry 50 may determine whether ventricular capture includes LBBAP or septal pacing based on a morphological metric. In some examples, processing circuitry 50 may determine the morphological metric by comparing, for each of cardiac cycles 122, a corresponding analysis window to a template. For example, IMD 10 may collect EGM 120 for sensed cardiac cycles 122 and compare each of cardiac cycles 122 in EGM 120 to a template. Based on the comparison, processing circuitry 50 may output a morphological metric value, such as the match metric value shown in FIG. 5. The morphological metric may have a range from 0% to 100%, and the match metric value may indicate the similarity between a respective ventricular pacing beat to the template. For example, a match metric value of 100% may represent a near identical match and values less than 100% may represent correspondingly greater disparities between the two waveforms. A cardiac cycle with a relatively large match metric (e.g., higher similarities between the waveforms) may indicate a higher likelihood that ventricular capture includes LBBAP, while a cardiac cycle with a relatively small match metric (e.g., lower similarities between the waveforms) may indicate a lower likelihood that ventricular capture includes LBBAP.

In some examples, determining the similarity between cardiac cycles 122 and the template may entail using techniques such as Wavelet transforms to perform a time-frequency decomposition. An example wavelet transform algorithm may involve computing wavelet transform coefficients for cardiac cycles 122 and extracting the coefficients that describe the salient features of the waveforms associated with cardiac cycles 122. Processing circuitry 50 may compare coefficients of cardiac cycles 122 with coefficients of the template to compute corresponding match metric values for cardiac cycles 122. For details regarding computation of a match metric value, reference is made to U.S. Pat. No. 7,826,893 patent and U.S. Pat. No. 7,706,869. Briefly, if the wavelet coefficient numbers match and the coefficients have similar absolute amplitude, then a match weight for the coefficient is added to determine a match metric value.

A relatively large match metric value may indicate a greater similarity between a cardiac cycle and the template than a relatively small match metric value. Thus, in the example of FIG. 5, cardiac cycle 122A, which has a match metric value of 100, may be more similar to the template than cardiac cycle 122B, which has a match metric value of 82.

In some instances, processing circuitry 50 may determine whether ventricular capture includes LBBAP or septal pacing by determining whether the morphological metric satisfies a morphology condition. Processing circuitry 50 may determine that the morphological metric satisfies the morphology condition when the match metric value is equal to or greater than a threshold (e.g., 70%). As such, satisfaction of the morphology condition may be indicative of substantially normal ECG morphology. In some examples, the threshold may be expressed in formats other than percentage (e.g., as an absolute value).

A relatively large match metric value may indicate a higher likelihood that ventricular capture includes LBBAP (but not septal pacing), and a relatively small match metric value may indicate a higher likelihood that ventricular capture includes septal pacing (but not LBBAP). At the same time, a relatively small ventricular activation time metric value may indicate a higher likelihood that ventricular capture includes LBBAP (but not septal pacing), and a relatively large ventricular activation time metric may indicate that ventricular capture includes septal pacing (but not LBBAP). As such, processing circuitry 50 may use an algorithm based on both the morphological metric (e.g., the match metric) and the ventricular activation time metric (e.g., t₄ and/or T4R) to determine whether ventricular capture includes LBBAP or septal pacing.

For example, responsive to determining that the morphological metric satisfies the morphology condition (e.g., the match metric value is equal to or greater than 70), indicating a higher likelihood that ventricular capture includes LBBAP, processing circuitry 50 may determine whether the ventricular activation time metric value is greater than a first threshold. An example first t₄ threshold may be 350 ms, and an example first T4R threshold may be 1.5. However, other thresholds are contemplated by this disclosure. The ventricular activation time metric (being greater than the first ventricular activation time threshold may be indicative of a lower likelihood that ventricular capture includes LBBAP and instead likely includes septal pacing.

Conversely, responsive to determining that the morphological metric does not satisfy the morphology condition, indicating a lower likelihood that ventricular capture includes LBBAP, processing circuitry 50 may determine whether the ventricular activation time metric value is greater than a second threshold. The ventricular activation time metric value being greater than the second threshold may be indicative of septal pacing. The first threshold may be larger than the second threshold. This may be because when the morphological metric satisfies the morphology condition, which weighs in favor of a determination that ventricular capture includes LBBAP, a larger ventricular activation time is warranted to reverse that determination and for processing circuitry 50 to instead determine that ventricular capture includes septal pacing. An example second t₄ threshold may be 200 ms, and an example second T4R threshold may be 1.1.

Processing circuitry 50 may use the techniques of this disclosure to determine whether ventricular capture includes LBBAP or septal pacing in various scenarios. For example, processing circuitry 50 may determine whether ventricular capture includes LBBAP or septal pacing when performing initial capture detection. IMD 10 may perform initial capture detection by delivering a stimulation voltage having an initial magnitude (e.g., 5V) that ensures ventricular capture of LBBAP. Processing circuitry 50 may collect one or more templates during the initial capture detection (e.g., when IMD 10 is delivering a stimulation voltage having an initial magnitude that ensures ventricular capture of LBBAP). In some examples, processing circuitry 50 may run a cross-comparison of the collected templates to ensure fit.

IMD 10 may progressively decrease (e.g., step-down) the stimulation voltage from the initial magnitude. IMD 10 may do this when performing initial capture detection. For each cardiac cycle, processing circuitry 50 may determine whether ventricular capture includes LBBAP or septal pacing using the techniques of this disclosure. When the stimulation voltage decreases to a certain voltage threshold, ventricular capture may transition from LBBAP to septal pacing (e.g., because the magnitude of the stimulation voltage is now insufficient to induce LBBAP).

In another scenario, IMD 10 may provide therapy to a patient by delivering stimulation pulses at a set (e.g., predetermined, constant, etc.) voltage. Processing circuitry 50 may determine whether ventricular capture includes LBBAP or septal pacing using the techniques of this disclosure to monitor LBBAP therapy delivery and whether LBBAP therapy is being appropriately maintained for the patient.

In any case, processing circuitry 50 may perform at least one action based on determining whether ventricular capture includes LBBAP or septal pacing. For instance, processing circuitry 50 may output, based on the determinations for a plurality of paced heartbeats a report comprising an indication of whether ventricular capture transitioned between LBBAP and septal pacing.

For example, with reference to FIG. 5, processing circuitry 50 may determine that cardiac cycle 122A has a match metric value of 100 and a T4R value of 1, cardiac cycle 122B has a match metric value of 82 and a T4R value of 1.05, cardiac cycle 122C has a match metric value of 82 and a T4R value of 1.05, cardiac cycle 122D has a match metric value of 88 and a T4R value of 1.06, cardiac cycle 122E has a match metric value of 76 and a T4R value of 1.06, cardiac cycle 122F has a match metric value of 52 and a T4R value of 1.21, cardiac cycle 122G has a match metric value of 52 and a T4R value of 1.18, and cardiac cycle 122H has a match metric value of 49 and a T4R value of 1.19. In this example, the morphology condition threshold may be 70, the first ventricular activation time threshold may be a T4R value of 1.17, and the second ventricular activation time threshold may be a T4R value of 1.03. Accordingly, processing circuitry 50 may determine that cardiac cycles 122A-122E demonstrate ventricular capture that includes LBBAP (e.g., indicated by the “Algo” value of 2 in FIG. 5) and that cardiac cycles 122F-122H demonstrate ventricular capture that includes septal pacing (e.g., indicated by the “Algo” value of 3 in FIG. 5).

In the example of FIG. 5, ventricular capture transitioned between LBBAP and septal pacing at cardiac cycle 122F. Thus, in this example, processing circuitry 50 may output an indication indicating that ventricular capture transitioned between LBBAP and septal pacing, as well as that the transition occurred at cardiac cycle 122F. The report may also include the threshold voltage or voltage range that indicated the between LBBAP and septal pacing or suggest a pacing voltage to provide LBBAP capture.

Additionally or alternatively, processing circuitry 50 may modify a magnitude (e.g., voltage) of the ventricular pacing (in this way reestablishing or maintaining appropriate LBBAP therapy) in response to detecting the likely loss of LBBAP or detecting septal pacing. For instance, responsive to determining that ventricular capture includes septal pacing, processing circuitry 50 may increase the stimulation voltage to a magnitude that produces LBBAP capture. For example, if a transition from LBBAP to septal pacing occurs when the stimulation voltage decrements from 1.75 V to 1.5 V, processing circuitry 50 may increase the pacing voltage (e.g., to 1.75V or a greater stimulation voltage) within appropriate limits.

In various cases, particularly at low pacing magnitudes, IMD 10 may not sense a pacing pulse within a cardiac cycle, resulting from a loss of capture. In the event of loss of capture, the capture sense time (t_{c}) may be relatively large. For example, the the for cardiac cycle 122I is 223 ms, which is relatively large compared to the the for cardiac cycle 122A of 105 ms. As used herein, the may refer to the time from delivery of a stimulation voltage (Vₚ) to the negative deflection (e.g., a minimum value) of the far-field EGM within a sampling window (having a length of, e.g., 250 ms) from Vₚ. In some examples, t_{c} may refer to the time from delivery of a stimulation voltage (Vₚ) to a negative deflection having a magnitude equal to or greater than a threshold, such as 0.5 mV, 1 mV, etc. In some examples, responsive to the capture sense time for a cardiac cycle being equal to or greater than a capture sense time threshold (e.g., a t_{c} greater than 150 ms), processing circuitry 50 may determine that loss of capture has occurred and that LBBAP or septal pacing is indeterminate (e.g., indicated by the “Algo” value of 0 in FIG. 5).

Although described herein as being performed by processing circuitry 50 of IMD 10 and with respect to cardiac EGM 120, some or all of the techniques may be performed by processing circuitry of another device (e.g., processing circuitry 80, processing circuitry 98, etc.) and with respect to any cardiac EGM in which one or more heartbeats can be identified.

FIG. 6 is a conceptual diagram of a depolarization waveform 124. As shown in FIG. 6, IMD 10 delivers a pacing voltage (Vpace) to the patient at a first time 126. At a second time 128, depolarization waveform 124 achieves a maximum amplitude. Processing circuitry 50 may determine a maximum ventricular amplitude time 130 (e.g., tₘₐₓ) by subtracting second time 128 from first time 126. Processing circuitry 50 may determine a center of area 132 (e.g., Xₐ) under a segment 125 of depolarization waveform 124 using the center of area equation described above. Processing circuitry 50 may then determine the ventricular activation time metric (e.g., t₄) based on maximum ventricular amplitude time 130 and center of area 132 of depolarization waveform 124. For example, processing circuitry 50 may determine the ventricular activation time metric (e.g., t₄) by subtracting center of area 132 from maximum ventricular amplitude time 130 (e.g., tₘₐₓ−Xₐ).

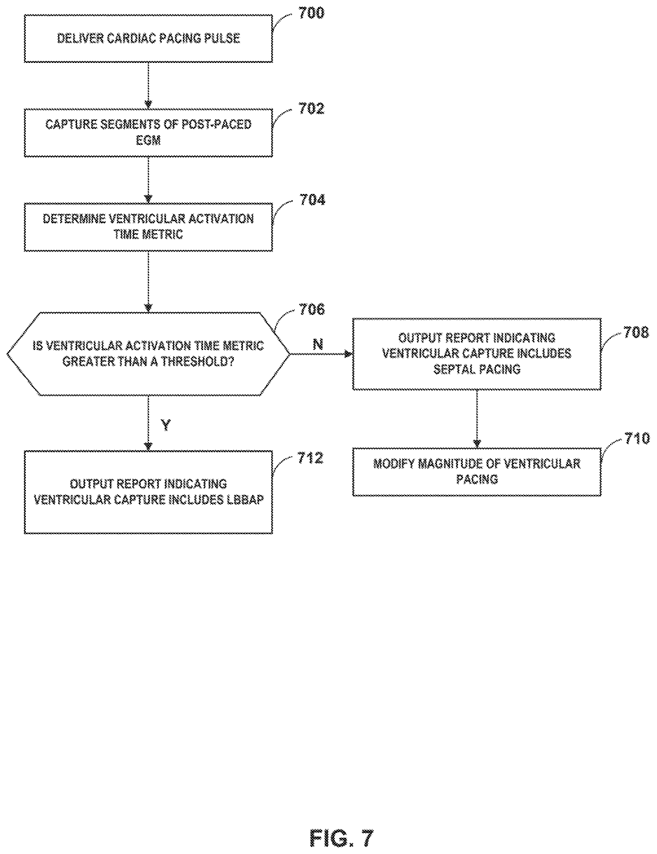

FIG. 7 is a flow diagram illustrating an example operation for detecting ventricular capture transitioning between LBBAP and septal pacing in accordance with techniques of this disclosure. Although the example operation of FIG. 7 is described as being performed by processing circuitry 50 of IMD 10 and explained based on select features of cardiac EGM 120 of FIG. 5, the observed pacing may occur at a single pacing voltage and not undergo the step-down voltage being shown in FIG. 5. Additionally, in other examples some or all of the example operation may be performed by processing circuitry of another device and with respect to any cardiac EGM.

IMD 10 may deliver a cardiac pacing pulse, e.g., to a ventricular septum and intended to provide LBBAP (700). IMD 10 may capture segments of post-paced EGMs, (e.g., within analysis windows 123) and then determine any EGM changes beat-by-beat (702). Processing circuitry 50 may determine a ventricular activation time metric (704) based on cardiac EGM 120, e.g., within analysis window 123. In general, a relatively low ventricular activation time metric value (e.g., a relatively low t₄ and/or T4R value) may indicate that ventricular capture includes LBBAP, and a relatively high ventricular activation time metric value may indicate that ventricular capture includes septal pacing (but not LBBAP).

Processing circuitry 50 may determine whether the ventricular activation time metric (e.g., t₄ and/or T4R) is greater than a ventricular activation time threshold (706). Responsive to determining that t₄ and/or T4R values are above respective ventricular activation time thresholds (“Y” of 706), processing circuitry 50 may output a report that ventricular capture includes septal pacing (708). In some examples, processing circuitry 50 may modify (e.g., increase) the stimulation voltage in response to ventricular capture including septal pacing (710). On the other hand, responsive to determining that t₄ and/or T4R values are below respective ventricular activation time thresholds (“N” of 706), processing circuitry 50 may output a report that ventricular capture includes LBBAP (712).

The example technique of FIG. 7 may be implemented as part of any known cardiac pacing threshold determination technique, which may occur during implantation of IMD 10 or during a clinic visit as directed by external device 12, or may occur automatically on a scheduled or periodic basis. Although described as being performed for a single cardiac beat or cycle after delivery of a ventricular pacing pulse intended to provide LBBAP, the example technique of FIG. 7 may be performed a plurality of times at a given cardiac pacing magnitude before processing circuitry determines whether that pacing magnitude results in LBBAP or septal pacing. In some examples, the example technique of FIG. 7 may be performed during delivery of pacing at multiple magnitudes to search for a magnitude at which capture transitions between LBBAP and septal pacing. The processing circuitry may report the transition magnitude, e.g., amplitude, and/or determine a magnitude for subsequent pacing operation, e.g., by adding a margin of safety to the determined transition amplitude.

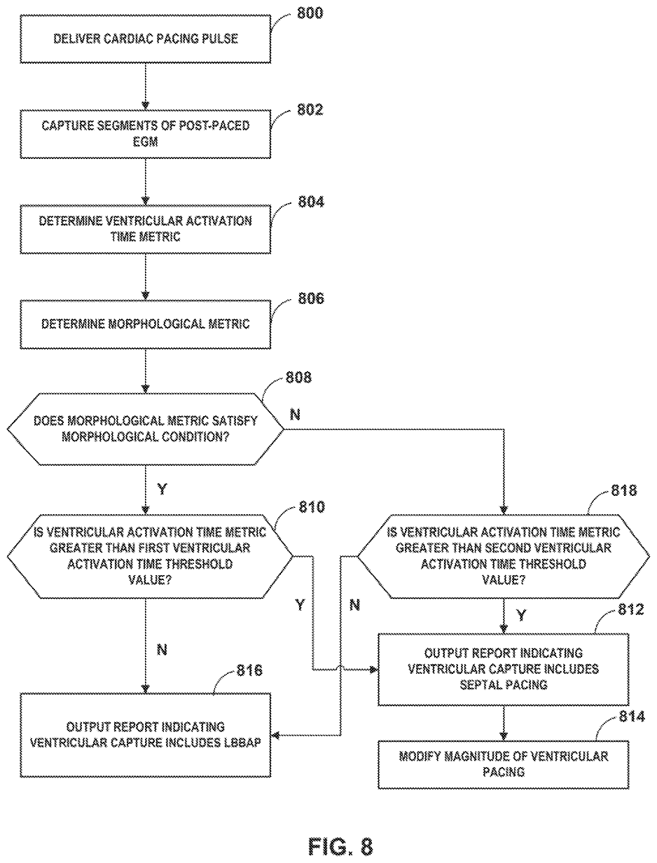

FIG. 8 is a flow diagram illustrating an example operation for detecting ventricular capture transitioning between LBBAP and septal pacing in accordance with techniques of this disclosure. Although the example operation of FIG. 8 is described as being performed by processing circuitry 50 of IMD 10 and explained based on select features of cardiac EGM 120 of FIG. 5, the observed pacing may occur at a single pacing voltage and not undergo the step-down voltage being shown in FIG. 5. Additionally, in other examples some or all of the example operation may be performed by processing circuitry of another device and with respect to any cardiac EGM.

IMD 10 may deliver a cardiac pacing pulse, e.g., to a ventricular septum and intended to provide LBBAP (800). IMD 10 may capture segments of post-paced EGMs, (e.g., within analysis windows 123) and then determine any EGM changes beat-by-beat (802). Processing circuitry 50 may determine a ventricular activation time metric (804) based on cardiac EGM 120, e.g., within analysis window 123. In general, a relatively low ventricular activation time metric value (e.g., a relatively low t₄ and/or T4R value) may indicate that ventricular capture includes LBBAP, and a relatively high ventricular activation time metric value may indicate that ventricular capture includes septal pacing (but not LBBAP).

In some examples, processing circuitry 50 may determine a morphological metric (806). Processing circuitry 50 may determine the morphologic metric by comparing the EGM 120 within analysis window 123 to a template waveform representing confirmed LBBAP. Based on the comparison, processing circuitry 50 may output a morphological metric. In some examples, processing circuitry 50 may use a Wavelet transform algorithm to compare coefficients of cardiac cycles 122 with coefficients of the template waveform to compute match metric values for cardiac cycles 122.

Processing circuitry 50 may determine whether the morphological metric satisfies a morphology condition (808). For example, the morphological metric may satisfy the morphology condition when the match metric value is equal to or greater than a threshold (e.g., 70). The threshold may be an absolute value, a percentage, etc. Satisfaction of the morphology condition may be indicative of a higher likelihood of LBBAP.

Responsive to determining that the morphological metric satisfies the morphology condition (“Y” of 808), processing circuitry 50 may determine whether the ventricular activation time metric value is greater than a first ventricular activation time threshold (810). Responsive to determining that t₄ and/or T4R values are above respective first ventricular activation time thresholds (“Y” of 810), processing circuitry 50 may output a report that ventricular capture includes septal pacing (812). In some examples, processing circuitry 50 may modify (e.g., increase) the stimulation voltage in response to ventricular capture including septal pacing (814). On the other hand, responsive to determining that t₄ and/or T4R values are below respective first ventricular activation time thresholds (“N” of 810), processing circuitry 50 may output a report that ventricular capture includes LBBAP (816).

Responsive to determining that the morphological metric does not satisfy the morphology condition (“N” of 808), e.g., indicating possible departure from the intended therapeutic pacing, processing circuitry 50 may determine whether the ventricular activation time metric value is greater than a second ventricular activation time threshold (818). The first ventricular activation time threshold may be larger than the second ventricular activation time threshold due to the morphological metric indicating possible departure from the intended therapeutic pacing. Hence, the second ventricular activation time threshold is set at a level that provides more scrutiny. Example second ventricular activation time thresholds may include a t₄ value of 200 ms and/or a T4R value of 1.1, though other values are possible. Responsive to determining that the ventricular activation time metric value is greater than the second ventricular activation time threshold (“Y” of 818), processing circuitry 50 may output an indication indicating that ventricular capture includes septal pacing (812). If ventricular capture previously included LBBAP, processing circuitry 50 may output an indication indicating that ventricular capture transitioned between LBBAP and septal pacing. In some examples, in response to determining that ventricular capture includes septal pacing, processing circuitry 50 may modify a magnitude of the pacing voltage to induce a transition from septal pacing to LBBAP (e.g., by increasing the pacing voltage by a predetermined amount or to the last pacing voltage that coincided with ventricular capture including LBBAP, or running the threshold detection analysis (FIG. 5) to determine a new pacing voltage that includes LBBAP) (814).

Responsive to determining that the ventricular activation time metric is not greater than the second ventricular activation time threshold (“N” of 818), processing circuitry 50 may output an indication indicating that ventricular capture includes LBBAP or the metric results (816). If ventricular capture previously included LBBAP, processing circuitry 50 may output an indication indicating that ventricular capture did not transition between LBBAP and septal pacing.

The example technique of FIG. 8 may be implemented as part of any known cardiac pacing threshold determination technique, which may occur during implantation of IMD 10 or during a clinic visit as directed by external device 12, or may occur automatically on a scheduled or periodic basis. Although described as being performed for a single cardiac beat or cycle after delivery of a ventricular pacing pulse intended to provide LBBAP, the example technique of FIG. 8 may be performed a plurality of times at a given cardiac pacing magnitude before processing circuitry determines whether that pacing magnitude results in LBBAP or septal pacing. In some examples, the example technique of FIG. 8 may be performed during delivery of pacing at multiple magnitudes to search for a magnitude at which capture transitions between LBBAP and septal pacing. The processing circuitry may report the transition magnitude, e.g., amplitude, and/or determine a magnitude for subsequent pacing operation, e.g., by adding a margin of safety to the determined transition amplitude.

This disclosure includes various examples, such as the following examples.

Example 1: A medical device system includes a medical device configured to deliver ventricular pacing to a heart of a patient; and processing circuitry configured to: receive cardiac electrogram data of a patient sensed by the medical device during delivery of ventricular pacing; identify a plurality of paced heartbeats within the cardiac electrogram data; for each of the plurality of heartbeats: determine a ventricular activation time metric; and determine, based on the ventricular activation time metric, whether ventricular capture includes left bundle branch area pacing or septal pacing; and responsive to determining ventricular capture includes septal pacing, modify a magnitude of the ventricular pacing.

Example 2: The medical device system of example 1, wherein the processing circuitry is configured to determine the ventricular activation time metric for each heartbeat of the plurality of heartbeats by: determining a center of area under a segment of a depolarization waveform and a maximum ventricular amplitude time for the heartbeat; and determining the ventricular activation time metric for the heartbeat based on the center of area and the maximum ventricular amplitude time.

Example 3: The medical device system of example 2, wherein the processing circuitry is configured to determine the ventricular activation time metric by subtracting the center of area from the maximum ventricular amplitude time.

Example 4: The medical device system of any of examples 1 to 3, wherein the processing circuitry is further configured to: determine a morphological metric; and determine, based on the morphological metric and the ventricular activation time metric, whether ventricular capture includes left bundle branch area pacing or septal pacing.

Example 5: The medical device system of example 4, wherein the processing circuitry is configured to determine the morphological metric by: comparing, for each of the plurality of heartbeats, a corresponding analysis window to a template; and determining the morphological metric based on the comparison.

Example 6: The medical device system of example 5, wherein the processing circuitry is configured to compare the corresponding analysis window to the template by using a wavelet transform algorithm.

Example 7: The medical device system of any of examples 4 to 6, wherein the processing circuitry is configured to determine whether ventricular capture includes left bundle branch area pacing or septal pacing by determining whether the morphological metric satisfies a morphology condition.

Example 8: The medical device system of example 7, wherein satisfaction of the morphology condition is indicative of a higher likelihood of left bundle branch area pacing.

Example 9: The medical device system of example 7 or 8, wherein the processing circuitry is further configured to: responsive to determining that the morphological metric satisfies the morphology condition, determine whether the ventricular activation time metric value is greater than a first ventricular activation time threshold, wherein the ventricular activation time metric value being greater than the first ventricular activation time threshold is indicative of septal pacing.

Example 10: The medical device system of any of examples 7 to 9, wherein the processing circuitry is further configured to: responsive to determining that the morphological metric does not satisfy the morphology condition, determine whether the ventricular activation time metric value is greater than a second ventricular activation time threshold, wherein the ventricular activation time metric value being greater than the second ventricular activation time threshold is indicative of septal pacing.

Example 11: The medical device system of any of examples 1 to 10, wherein the processing circuitry is configured to modify the magnitude of the ventricular pacing by increasing a stimulation voltage.

Example 12: The medical device system of any of examples 1 to 11, wherein the processing circuitry is configured to modify the magnitude of the ventricular pacing to reestablish left bundle branch area pacing.

Example 13: The medical device system of any of examples 1 to 12, wherein the processing circuitry is further configured to output a report including an indication of whether ventricular capture includes left bundle branch area pacing or septal pacing.

Example 14: The medical device system of any of examples 1 to 13, wherein the medical device includes the processing circuitry.

Example 15: A method includes receiving, by processing circuitry of a medical device system includes determining, by the processing circuitry, a ventricular activation time metric; and determining, by the processing circuitry and based on the ventricular activation time metric, whether ventricular capture includes left bundle branch area pacing or septal pacing; and responsive to determining ventricular capture includes septal pacing, modifying, by the processing circuitry, a magnitude of the ventricular pacing.

Example 16: The method of example 15, wherein determining the ventricular activation time metric for each heartbeat of the plurality of heartbeats includes: determining, by the processing circuitry, a center of area under a segment of a depolarization waveform and a maximum ventricular amplitude time for the heartbeat; and determining the ventricular activation time metric for the heartbeat based on the center of area and the maximum ventricular amplitude time.

Example 17: The method of example 16, wherein determining the ventricular activation time metric includes subtracting the center of area from the maximum ventricular amplitude time.

Example 18: The method of any of examples 15 to 17, further includes determining, by the processing circuitry, a morphological metric; and determining, by the processing circuitry and based on the morphological metric and the ventricular activation time metric, whether ventricular capture includes left bundle branch area pacing or septal pacing.

Example 19: The method of example 18, wherein determining the morphological metric includes: comparing, for each of the plurality of heartbeats, a corresponding analysis window to a template; and determining the morphological metric based on the comparison.

Example 20: The method of example 19, wherein comparing the corresponding analysis window to the template includes using a wavelet transform algorithm.

Example 21: The method of any of examples 18 to 20, wherein determining whether ventricular capture includes left bundle branch area pacing or septal pacing includes determining whether the morphological metric satisfies a morphology condition.

Example 22: The method of example 21, wherein satisfaction of the morphology condition is indicative of a higher likelihood of left bundle branch area pacing.

Example 23: The method of example 21 or 22, further including, responsive to determining that the morphological metric satisfies the morphology condition, determining whether the ventricular activation time metric value is greater than a first ventricular activation time threshold, wherein the ventricular activation time metric value being greater than the first ventricular activation time threshold is indicative of septal pacing.

Example 24: The method of any of examples 21 to 23, further including, responsive to determining that the morphological metric does not satisfy the morphology condition, determining whether the ventricular activation time metric value is greater than a second ventricular activation time threshold, wherein the ventricular activation time metric value being greater than the second ventricular activation time threshold is indicative of septal pacing.

Example 25: The method of any of examples 15 to 25, further including outputting, by the processing circuitry and based on the determinations for the plurality of heartbeats, a report including an indication of whether ventricular capture includes left bundle branch area pacing or septal pacing.

Example 26: The method of example 1, further including progressively decreasing, from an initial magnitude, a stimulation voltage of the ventricular pacing to perform initial capture detection.

Example 27: An implantable medical device includes therapy delivery circuitry configured to deliver ventricular pacing to a heart of a patient; sensing circuitry configured to sense a cardiac electrogram data of the patient during delivery of ventricular pacing; processing circuitry configured to: identify a plurality of heartbeats within the cardiac electrogram data; for each of the plurality of heartbeats: determine a ventricular activation time metric; and determine, based on the ventricular activation time metric, whether ventricular capture includes left bundle branch area pacing or septal pacing; and responsive to determining ventricular capture includes septal pacing, modify a magnitude of the ventricular pacing.

Example 28: The implantable medical device of example 27, wherein the processing circuitry is configured to determine the ventricular activation time metric for each heartbeat of the plurality of heartbeats by: determining a center of area under a segment of a depolarization waveform and a maximum ventricular amplitude time for the heartbeat; and determining the ventricular activation time metric for the heartbeat based on the center of area and the maximum ventricular amplitude time.

Example 29: The implantable medical device of example 28, wherein the processing circuitry is configured to determine the ventricular activation time metric by subtracting the center of area from the maximum ventricular amplitude time.

Example 30: The implantable medical device of any of examples 27 to 29, wherein the processing circuitry is further configured to: determine a morphological metric; and determine, based on the morphological metric and the ventricular activation time metric, whether ventricular capture includes left bundle branch area pacing or septal pacing.

Example 31: The implantable medical device of example 30, wherein the processing circuitry is configured to determine the morphological metric by: comparing, for each of the plurality of heartbeats, a corresponding analysis window to a template; and determining the morphological metric based on the comparison.

Example 32: The implantable medical device of example 31, wherein the processing circuitry is configured to compare the corresponding analysis window to the template by using a wavelet transform algorithm.

Example 33: The implantable medical device of any of examples 30 to 32, wherein the processing circuitry is configured to determine whether ventricular capture includes left bundle branch area pacing or septal pacing by determining whether the morphological metric satisfies a morphology condition.

Example 34: The implantable medical device of example 33, wherein satisfaction of the morphology condition is indicative of a higher likelihood of left bundle branch area pacing.

Example 35: The implantable medical device of example 33 or 34, wherein the processing circuitry is further configured to: responsive to determining that the morphological metric satisfies the morphology condition, determine whether the ventricular activation time metric value is greater than a first ventricular activation time threshold, wherein the ventricular activation time metric value being greater than the first ventricular activation time threshold is indicative of septal pacing.

Example 36: The implantable medical device of any of examples 33 to 35, wherein the processing circuitry is further configured to: responsive to determining that the morphological metric does not satisfy the morphology condition, determine whether the ventricular activation time metric value is greater than a second ventricular activation time threshold, wherein the ventricular activation time metric value being greater than the second ventricular activation time threshold is indicative of septal pacing.

Example 37: The implantable medical device of any of examples 27 to 36, wherein the processing circuitry is configured to modify the magnitude of the ventricular pacing by increasing a stimulation voltage.

Example 38: The implantable medical device of any of examples 27 to 37, wherein the processing circuitry is configured to modify the magnitude of the ventricular pacing to reestablish left bundle branch area pacing.

Example 39: The implantable medical device of any of examples 27 to 38, wherein the processing circuitry is further configured to output a report including an indication of whether ventricular capture includes left bundle branch area pacing or septal pacing.

The techniques described in this disclosure may be implemented, at least in part, in hardware, software, firmware, or any combination thereof. For example, various aspects of the techniques may be implemented within one or more microprocessors, DSPs, ASICS, FPGAs, or any other equivalent integrated or discrete logic QRS circuitry, as well as any combinations of such components, embodied in external devices, such as physician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, DRAM, SRAM, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

1. A medical device system comprising:
a medical device configured to deliver ventricular pacing to a heart of a patient; and
processing circuitry configured to:
receive cardiac electrogram data of a patient sensed by the medical device during delivery of ventricular pacing;
identify a plurality of paced heartbeats within the cardiac electrogram data;
for each of the plurality of heartbeats:
determine a ventricular activation time metric; and
determine, based on the ventricular activation time metric, whether ventricular capture comprises left bundle branch area pacing or septal pacing; and
responsive to determining ventricular capture comprises septal pacing, modify a magnitude of the ventricular pacing.

2. The medical device system of claim 1, wherein the processing circuitry is configured to determine the ventricular activation time metric for each heartbeat of the plurality of heartbeats by:
determining a center of area under a segment of a depolarization waveform and a maximum ventricular amplitude time for the heartbeat; and
determining the ventricular activation time metric for the heartbeat based on the center of area and the maximum ventricular amplitude time.

3. The medical device system of claim 2, wherein the processing circuitry is configured to determine the ventricular activation time metric by subtracting the center of area from the maximum ventricular amplitude time.

4. The medical device system of claim 1, wherein the processing circuitry is further configured to:
determine a morphological metric; and
determine, based on the morphological metric and the ventricular activation time metric, whether ventricular capture comprises left bundle branch area pacing or septal pacing.

5. The medical device system of claim 4, wherein the processing circuitry is configured to determine the morphological metric by:
comparing, for each of the plurality of heartbeats, a corresponding analysis window to a template; and
determining the morphological metric based on the comparison.

6. The medical device system of claim 5, wherein the processing circuitry is configured to compare the corresponding analysis window to the template by using a wavelet transform algorithm.

7. The medical device system of claim 4, wherein the processing circuitry is configured to determine whether ventricular capture comprises left bundle branch area pacing or septal pacing by determining whether the morphological metric satisfies a morphology condition, wherein satisfaction of the morphology condition is indicative of a higher likelihood of left bundle branch area pacing.

8. The medical device system of claim 7, wherein the processing circuitry is further configured to:
responsive to determining that the morphological metric satisfies the morphology condition, determine whether the ventricular activation time metric value is greater than a first ventricular activation time threshold, wherein the ventricular activation time metric value being greater than the first ventricular activation time threshold is indicative of septal pacing.

9. The medical device system of claim 7, wherein the processing circuitry is further configured to:
responsive to determining that the morphological metric does not satisfy the morphology condition, determine whether the ventricular activation time metric value is greater than a second ventricular activation time threshold, wherein the ventricular activation time metric value being greater than the second ventricular activation time threshold is indicative of septal pacing.

10. The medical device system of claim 1, wherein the processing circuitry is configured to modify the magnitude of the ventricular pacing by increasing a stimulation voltage to reestablish left bundle branch area pacing.

11. A method comprising:
receiving, by processing circuitry of a medical device system comprising a medical device, cardiac electrogram data of a patient sensed by the medical device during delivery of ventricular pacing;
identifying, by the processing circuitry, a plurality of heartbeats within the cardiac electrogram data;
for each of the plurality of heartbeats:
determining, by the processing circuitry, a ventricular activation time metric; and
determining, by the processing circuitry and based on the ventricular activation time metric, whether ventricular capture comprises left bundle branch area pacing or septal pacing; and
responsive to determining ventricular capture comprises septal pacing, modifying, by the processing circuitry, a magnitude of the ventricular pacing.

12. The method of claim 11, wherein determining the ventricular activation time metric for each heartbeat of the plurality of heartbeats comprises:
determining, by the processing circuitry, a center of area under a segment of a depolarization waveform and a maximum ventricular amplitude time for the heartbeat; and
determining the ventricular activation time metric for the heartbeat based on the center of area and the maximum ventricular amplitude time.

13. The method of claim 12, wherein determining the ventricular activation time metric comprises subtracting the center of area from the maximum ventricular amplitude time.

14. The method of claim 11, further comprising:
determining, by the processing circuitry, a morphological metric; and
determining, by the processing circuitry and based on the morphological metric and the ventricular activation time metric, whether ventricular capture comprises left bundle branch area pacing or septal pacing.

15. The method of claim 14, wherein determining the morphological metric comprises:
comparing, for each of the plurality of heartbeats, a corresponding analysis window to a template; and
determining the morphological metric based on the comparison.

16. The method of claim 15, wherein comparing the corresponding analysis window to the template comprises using a wavelet transform algorithm.

17. The method of claim 14, wherein determining whether ventricular capture comprises left bundle branch area pacing or septal pacing comprises determining whether the morphological metric satisfies a morphology condition, wherein satisfaction of the morphology condition is indicative of a higher likelihood of left bundle branch area pacing.

18. The method of claim 17, further comprising, responsive to determining that the morphological metric satisfies the morphology condition, determining whether the ventricular activation time metric value is greater than a first ventricular activation time threshold, wherein the ventricular activation time metric value being greater than the first ventricular activation time threshold is indicative of septal pacing.

19. The method of claim 17, further comprising, responsive to determining that the morphological metric does not satisfy the morphology condition, determining whether the ventricular activation time metric value is greater than a second ventricular activation time threshold, wherein the ventricular activation time metric value being greater than the second ventricular activation time threshold is indicative of septal pacing.

20. The method of claim 11, further comprising outputting, by the processing circuitry and based on the determinations for the plurality of heartbeats, a report comprising an indication of whether ventricular capture includes left bundle branch area pacing or septal pacing.

21. The method of claim 11, further comprising progressively decreasing, from an initial magnitude, a stimulation voltage of the ventricular pacing to perform initial capture detection.

22. An implantable medical device comprising:
therapy delivery circuitry configured to deliver ventricular pacing to a heart of a patient;
sensing circuitry configured to sense a cardiac electrogram data of the patient during delivery of ventricular pacing;
processing circuitry configured to:
identify a plurality of heartbeats within the cardiac electrogram data;
for each of the plurality of heartbeats:
determine a ventricular activation time metric; and
determine, based on the ventricular activation time metric, whether ventricular capture comprises left bundle branch area pacing or septal pacing; and
responsive to determining ventricular capture comprises septal pacing, modify a magnitude of the ventricular pacing.

23. The implantable medical device of claim 22, wherein the processing circuitry is configured to determine the ventricular activation time metric for each heartbeat of the plurality of heartbeats by:
determining a center of area under a segment of a depolarization waveform and a maximum ventricular amplitude time for the heartbeat; and
determining the ventricular activation time metric for the heartbeat based on the center of area and the maximum ventricular amplitude time.

24. The implantable medical device of claim 23, wherein the processing circuitry is configured to determine the ventricular activation time metric by subtracting the center of area from the maximum ventricular amplitude time.

25. The implantable medical device of claim 22, wherein the processing circuitry is further configured to:
determine a morphological metric; and
determine, based on the morphological metric and the ventricular activation time metric, whether ventricular capture comprises left bundle branch area pacing or septal pacing.

26. The implantable medical device of claim 25, wherein the processing circuitry is configured to determine the morphological metric by:
comparing, for each of the plurality of heartbeats, a corresponding analysis window to a template; and
determining the morphological metric based on the comparison.

27. The implantable medical device of claim 26, wherein the processing circuitry is configured to compare the corresponding analysis window to the template by using a wavelet transform algorithm.

28. The implantable medical device of claim 25, wherein the processing circuitry is configured to determine whether ventricular capture comprises left bundle branch area pacing or septal pacing by determining whether the morphological metric satisfies a morphology condition, wherein satisfaction of the morphology condition is indicative of a higher likelihood of left bundle branch area pacing.

29. The implantable medical device of claim 28, wherein the processing circuitry is further configured to:
responsive to determining that the morphological metric satisfies the morphology condition, determine whether the ventricular activation time metric value is greater than a first ventricular activation time threshold, wherein the ventricular activation time metric value being greater than the first ventricular activation time threshold is indicative of septal pacing.

30. The implantable medical device of claim 28, wherein the processing circuitry is further configured to:
responsive to determining that the morphological metric does not satisfy the morphology condition, determine whether the ventricular activation time metric value is greater than a second ventricular activation time threshold, wherein the ventricular activation time metric value being greater than the second ventricular activation time threshold is indicative of septal pacing.

31. The implantable medical device of claim 22, wherein the processing circuitry is configured to modify the magnitude of the ventricular pacing by increasing a stimulation voltage to reestablish left bundle branch area pacing.
